(19) United States
(12) Patent Application Publication
(10) Pub. No.: US 2022/0375584 A1
(43) Pub. Date: Nov. 24, 2022

(54) SYSTEM AND METHOD FOR MANAGING MEDICAL DEVICES AND MEDICAL DEVICE CONSUMABLES

(71) Applicants: Mark T. McBride, Coronado, CA (US); Glenn Butler, Hollison, MA (US); John Roughneen, Dennis, MA (US)

(72) Inventors: Mark T. McBride, Coronado, CA (US); Glenn Butler, Hollison, MA (US); John Roughneen, Dennis, MA (US)

(21) Appl. No.: 17/741,990

(22) Filed: May 11, 2022

Related U.S. Application Data
(63) Continuation-in-part of application No. 63/190,841, filed on May 20, 2021.

(51) Int. Cl. G16H 40/40; G16H 40/67; G16H 40/20

(52) U.S. Cl. CPC G16H 40/40; G16H 40/67; G16H 40/20

(57) ABSTRACT

Systems, methods, and other embodiments associated with techniques for managing devices and/or consumables, such as medical devices and medical device consumables are described. In one embodiment, the method includes determining if a consumable has been properly installed, determining if the consumable is authentic, determining if the consumable is operating correctly, determining if the consumable needs to be replaced due to a time of usage, determining if the consumable needs to be re-ordered, determining if a user of the consumable needs to be billed for the use of the consumable, and obtaining information from the consumable regarding an operating characteristic of the consumable in order to provide feedback regarding the operational efficiency of the system.

### CROSS-REFERENCE TO RELATED APPLICATIONS

This application is a continuation-in-part of U.S. Provisional Patent Application 63/190,841, filed on May 20, 2021, the disclosure of which is hereby incorporated by reference in its entirety to provide continuity of disclosure to the extent such a disclosure is not inconsistent with the disclosure herein.

### FIELD OF THE INVENTION

The present invention is generally related to a system and method for managing medical devices and medical device consumables. The system would keep track of consumables used in the medical device such as HEPA filters, batteries, or the like along with other medical consumables such as surgical gowns. The system would periodically ship consumables to the user based upon the tracked usage of the consumables. The system would provide feedback to the user regarding the usage of the consumables. Furthermore, the system would provide information to the user about preventative maintenance of the medical device such as performance of a motor in the medical device. Still further, the system would provide feedback regarding the safety of using the consumable and/or the medical device and prevent the use of the medical device if the medical device might be unsafe due to an expired consumable or a consumable malfunction. Also, if the user does not own the medical device, the system would periodically send billing statements such as invoices to the user. Finally, the system would be able to collect information about the usage of the consumables and the medical devices for product improvement.

### BACKGROUND OF THE INVENTION

Prior to the present invention, as set forth in general terms above and more specifically below, it is known, that medical devices need to be properly installed and maintained in order to function properly. A medical device that is not properly installed or maintained may fail at a very inopportune time, which could lead to a serious incident during a medical operation or procedure. Furthermore, it is known that consumables are often used with medical devices. For example, motors, such as fan motors, are used with surgical hoods in order to circulate air that is being introduced into the surgical hood of the medical personnel performing a surgical procedure. If the motor is not properly maintained or installed or the batteries used to power the fan motor are not checked to make sure that they are capable of powering the fan motor and the fan motor and/or the batteries fail during the surgical procedure, this could result in a serious incident during the medical procedure. If it could be assured that the medical device has been properly installed and maintained and is working properly, this should substantially reduce the likelihood that the medical device will malfunction during the medical procedure or operation. Also, if it could be assured that the consumables associated with the medical device were properly installed and maintained, this also should substantially reduce the likelihood that the medical device consumable will malfunction during the medical procedure or operation.

It is a purpose of this invention to fulfill these and other needs in the medical device and medical device consumables art in a manner more apparent to the skilled artisan once given the following disclosure.

The preferred system and method for managing medical devices and medical device consumables, according to various embodiments of the present invention, offers the following advantages: ease of use; the ability to keep track of medical device installation, usage and maintenance; the ability to keep track of medical device consumable installation, usage and maintenance; the ability to provide billing statements for medical device and medical device consumable usage; the ability to provide feedback regarding the medical device usage and the medical device consumable usage for preventative maintenance; the ability to collect information regarding the medical device usage and the medical device consumable usage for product improvement; the ability to provide audible and haptic (touch) alerts; and the ability to be able to ship, in a timely manner, the needed medical device and/or medical device consumable to the user. In fact, in many of the preferred embodiments, these advantages are optimized to an extent that is considerably higher than heretofore achieved in prior, known systems and methods for managing medical devices and medical device consumables.

### BRIEF DESCRIPTION OF THE DRAWINGS

The above-mentioned features and steps of the invention and the manner of attaining them will become apparent, and the invention itself will be best understood by reference to the following description of the embodiments of the invention in conjunction with the accompanying drawings, wherein like characters represent like parts throughout the several views and in which:

FIG. 1 is a schematic, isometric view of a helmetless support for use with surgical hoods and gowns, according to one embodiment of the present invention;

FIG. 2 is a schematic, isometric view of a ventilation system for use with surgical hoods and gowns, constructed according to an embodiment of the present invention;

FIG. 3 is a schematic, side view of the ventilation system for use with surgical hoods and gowns illustrating the adjustable face vent air flow levers, constructed according to an embodiment of the present invention;

FIG. 4 is a cut-away view, taken along lines 4-4 of FIG. 3 of the ventilation system for use with surgical hoods and gowns illustrating the adjustable face vent air flow levers, constructed according to an embodiment of the present invention;

FIG. 5 is a schematic, isometric, back view of the ventilation system for use with surgical hoods and gowns with the back cover removed illustrating the power module, constructed according to an embodiment of the present invention;

FIG. 6 is an isometric, back view of the ventilation system for use with surgical hoods and gowns with the back cover removed illustrating the printed circuit board (PCB) module, constructed according to an embodiment of the present invention;

FIG. 7 illustrates one embodiment for a method for managing medical devices and medical device consumables;

FIG. 8 illustrates one embodiment for a method for determining if the consumable or device has been properly installed;

FIG. 9 illustrates one embodiment for a method for determining if the consumable or device is authentic;

FIG. 10 illustrates one embodiment for a method for determining if the device or consumable is operating correctly;

FIG. 11 illustrates one embodiment for a method for determining if the device or consumable needs to be replaced due to time of usage;

FIG. 12 illustrates one embodiment for a method for determining if the device or consumable needs to be re-ordered;

FIG. 13 illustrates one embodiment for a method for determining if the client needs to be billed for use of the device or consumable;

FIG. 14 illustrates one embodiment for a method for providing feedback to the system regarding the performance of the device or consumable; and

FIG. 15 illustrates an embodiment of a special purpose computing system configured with the example systems and/or methods disclosed.

### DETAILED DESCRIPTION OF THE PREFERRED EMBODIMENTS OF THE INVENTION

In order to address the shortcomings of the prior, known systems and methods for managing medical device maintenance and medical device consumables, it would be desirable to utilize a system and method for managing medical devices and medical device consumables. The system would keep track of consumables used in the medical device such as HEPA filters, batteries, or the like along with other medical consumables such as surgical gowns. The system would periodically ship consumables to the user based upon the tracked usage of the consumables. The system would provide feedback to the user regarding the usage of the consumables. Furthermore, the system would provide information to the user about preventative maintenance of the medical device such as performance of a motor in the medical device. Also, if the user does not own the medical device, the system would periodically send billing statements such as invoices to the user. Finally, the system would be able to collect information about the usage of the consumables and the medical devices for product improvement.

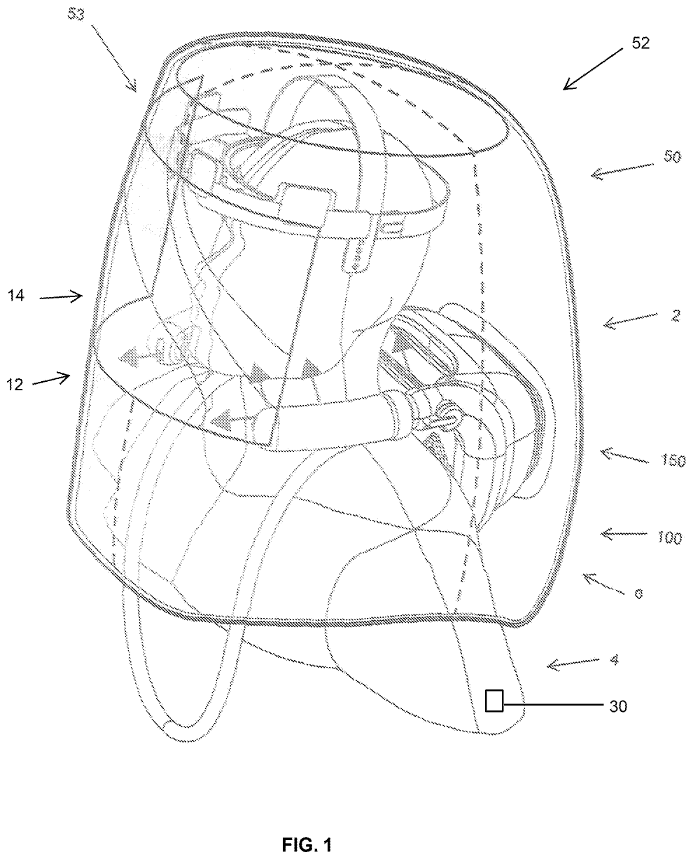

Referring now to FIG. 1-6, there is illustrated a helmetless support system 2 for use with surgical hoods and gowns. The helmetless support system 2 for use with surgical hoods and gowns can be used to support the one-piece surgical gown 4 and the surgical hood 14 without the need for the wearer 6 to wear a helmet. In this manner, one-piece surgical gown 4 and the surgical hood 14 completely and sterilely covers the head, neck, and torso of the wearer 6 when donned by the wearer 6. Also, the one-piece surgical gown 4 and the surgical hood 14 includes a clear faceplate 12. The helmetless support 2 further includes a helmetless surgical hood and gown support having a flexible headband 52 with attached lightweight front offsets 53 in front that can be releasably attached to the faceplate 12. Furthermore, the front offsets 53 are used to provide for air circulation around head of the wearer 6.

### Helmetless Surgical Hood and Gown Support

As shown in FIG. 1, helmetless support 2 for use with surgical hoods and gowns includes, in part, surgical gown 4, wearer 6, gown wireless identification system 30, and helmetless surgical hood and gown support 50. It is to be understood that surgical gown 4 is constructed of any suitable, durable, medical grade material. It is to be further understood that the surgical gown 4 is to be constructed into a one-piece design that will completely and sterilely cover the wearer when attached to the hood 14. Finally, it is to be understood that gown wireless identification system 30 is a conventional wireless identification system such as an RFID tag that can be conventionally attached to the surgical gown 4.

With respect to helmetless surgical hood and gown support 50, helmetless surgical hood and gown support 50 includes, in part, flexible, adjustable band 52 and front offsets 53. Preferably, flexible band 52 is constructed of any suitable, durable, flexible, medical grade material. An important feature of flexible band 52 being that it comfortably fits around the head of the wearer 6, but still is capable of securely holding surgical gown 4 and surgical hood 14 once the surgical gown 4 and surgical hood 14 have been attached to helmetless surgical hood and gown support 50 and then placed over the wearer, as will be discussed in greater detail later. In particular, it is important that flexible band 52 be able to securely hold hood 14 off of the head of wearer 6 and allow the air to flow around the head of wearer 6, as will be discussed in greater detail later.

### Helmetless Surgical Hood and Gown Ventilation System

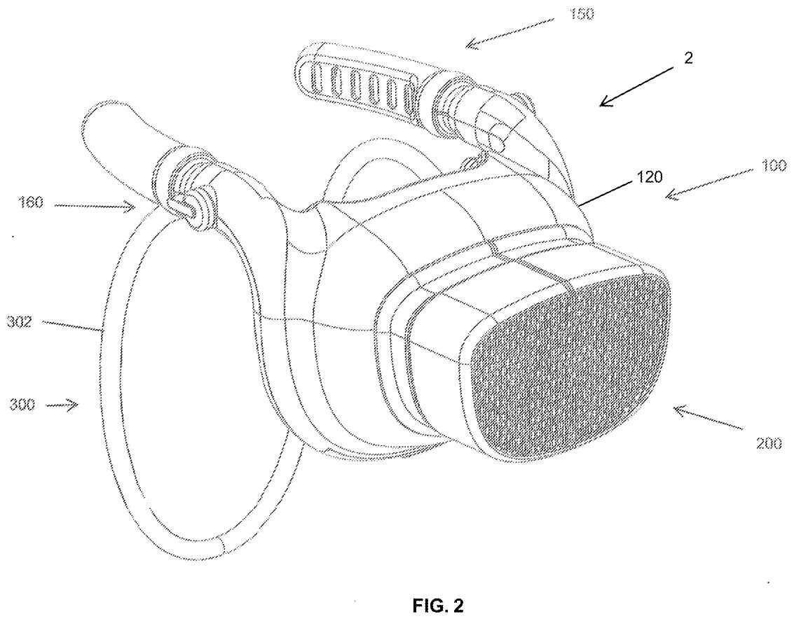

Referring now to FIGS. 2-6, there is illustrated a ventilation system 100 for use with helmetless support system 2. The ventilation system 100 is constructed such that the wearer 6 can control the fan speed once the gown 4 and hood 14 have been donned. A face vent module 150 is used as a “yoke” to support the ventilation system 100 on the shoulders of the wearer 6 (FIG. 1). Finally, the wearer 6 can control the output from each of the various output apertures (face vent module 150, and neck vent module 350) in the ventilation system 100, as will be discussed in greater detail later.

As shown in FIGS. 2-6, helmetless support system 2 for use with surgical hoods and gowns having ventilation system 100 includes, in part, protective casing 120, face vent module 150, air filtration module 200, power module 250, yoke module 300, neck vent module 350, air flow generation module 450, and printed circuit board (PCB) module 500.

A unique aspect of the present invention is the location of the ventilation system with respect to the surgical gown 4 and surgical hood 14. As shown in FIG. 1, the ventilation system 100 is almost completely located inside of the surgical gown 4 and surgical hood 14.

### Protective Casing 120

With respect to protective casing 120, protective casing 120, preferably, is constructed of any suitable, durable, high strength, shock resistant, UV resistant, medical grade polymeric material. It is to be understood that protective casing 120 is used to encase ventilation system 100 in order to provide protection for air filtration module 200, power module 250, neck vent module 350, air flow generation module 450, and printed circuit board (PCB) module 500.

### Face Vent Module 150

Regarding face vent module 150, as shown in FIGS. 2-6, face vent module 150, includes, in part, removable face vents 152, face vent openings 154, face vent connectors 156, face vent adaptors 158, face vent air flow adjustors 160, and face vent air flow adjuster lever 162. Preferably, face vents 152 and face vent connectors 156 are constructed as a single-piece construction and are constructed of any suitable, durable, lightweight, medical grade, and washable material. Also, face vent openings 154 are formed in removable face vents 152 by conventional techniques such as forming, stamping, molding, or the like. Face vent adaptors 158, preferably, are constructed of any suitable, durable, high strength, medical grade material and are permanently connected to protective casing 120 near face vent air flow adjustors 160 and face vent air flow adjuster levers 162. Finally, face vent air flow adjustors 160 and face vent air flow adjuster lever 162, preferably, are constructed of any suitable, durable, high strength, medical grade material.

A unique aspect of the present invention is the use of removable face vents 152. In particular, removable face vents 152 are constructed in such a manner that allows the removable face vents 152 to be easily removed from the face vent adaptors 158 so that the removable face vents 152 can be cleaned, disinfected, and sanitized prior to the next usage of the helmetless support 2 for use with surgical hoods and gowns having ventilation system 100. Once the removable face vents 152 have been cleaned, disinfected, and sanitized, the removable face vents 152 can be easily slid onto the face vent adaptors 158 by locating the face vent connectors 156 on the face vent adaptors 158.

A further unique aspect of the present invention is the use of face vent air flow adjustors 160 and face vent air flow adjuster lever 162. In particular, the wearer 6 can adjust the amount of air flow that is being emitted out of the removable face vents 152 through the use of vent air flow adjustor 160 and face vent air flow adjuster lever 162. In this manner, the wearer 6 can conventionally manipulate face vent air flow adjuster lever 162 so that the amount of air flow is adjusted. For example, the wearer 6 may push/pull the face vent air flow adjuster lever 162 upwards which will cause the amount of air flow being emitted out of the removable face vents 152 to be reduced. Conversely, the wearer 6 may push/pull the face vent air flow adjuster lever 162 downwards which will cause the amount of air flow being emitted out of the removable face vents 152 to be increased.

### Air Filtration Module 200

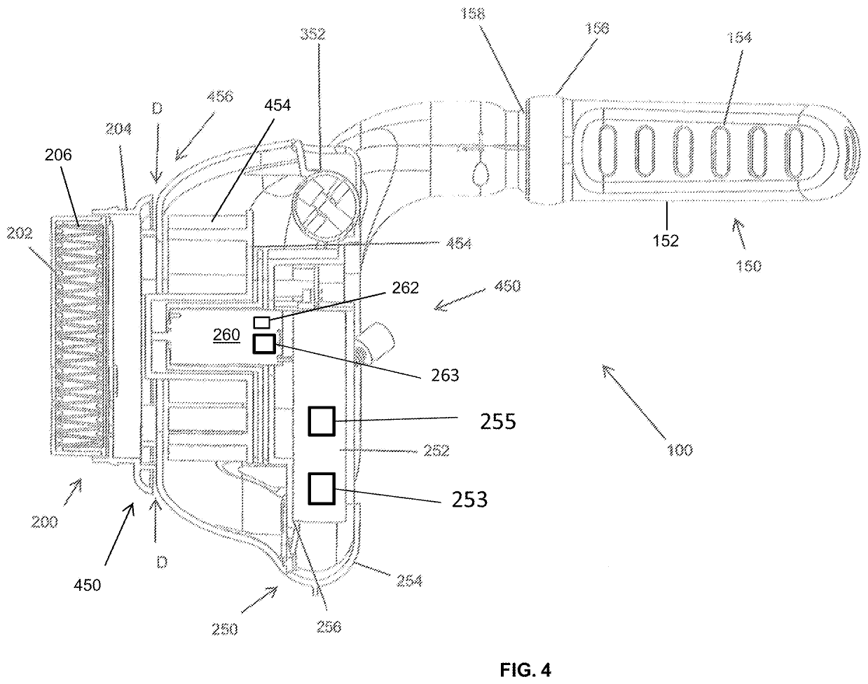

With respect to air filtration module 200, as shown in FIG. 4, air filtration module 200, includes, in part, air filter 202, air filtration adaptor 204, filter casing 206, and air filtration module wireless identification system 208. Preferably, air filter 202 is a HEPA (or ULPA) air filter that is located within a filter casing 206. Preferably, filter casing 206 is constructed of any suitable, durable, high strength, medical grade material. Preferably, air filtration adaptor 204 is conventionally formed on protective casing 120. Finally, it is to be understood that air filtration module wireless identification system 208 is a conventional wireless identification system such as an RFID tag that can be conventionally attached to or electrically connected to the air filter 202, as will be discussed in greater detail later.

A unique aspect of the present invention is the use of air filtration module 200. In particular, air filtration module 200 can be used to filter out air borne contaminants so that they do enter into the surgical hood 14 and surgical gown 4. As discussed above, only the air filter 202 extends outside of the surgical hood 14 (FIG. 1). In this manner, only air going through the air filtration module 200 will be allowed to enter into the surgical hood 14 and surgical gown 4. Also, the air filter 202 can be easily removed and replaced. For example, the wearer 6 can simply remove the air filter 202 and the filter casing 206 from the air filtration adaptor 204. The wearer 6 can then replace the used air filter 202 and filter casing 206 with a new air filter 202 and filter casing 206 by simply placing the new air filter 202 and filter casing 206 onto the air filtration adaptor 204. It is to be understood that the air filter 202 and filter casing 206 can be retained on the air filtration adaptor 204 by a snap fit, a threaded connection, a bayonet connection, a slidable connection or the like, as will be discussed in greater detail later.

### Power Module 250

Regarding power module 250, as shown in FIGS. 4-6, power module 250, includes, in part, battery 252, battery doors 254, battery lock 256, power module identification system 258, motor 260, and motor sensor/wireless identification system 262. Preferably, battery 252 is a conventional, rechargeable battery such as a lithium-ion battery or the like that is capable of providing sufficient power to air flow generation module 450 and printed circuit board (PCB) module 500 for an extended period of time such as 6-8 hours. Also, battery doors 254, preferably are constructed of any suitable, durable, high strength, medical grade material. It is to be understood that wireless identification system 258 is a conventional wireless identification system such as an RFID tag that can be conventionally attached to or electrically connected to the battery 252, as will be discussed in greater detail later. It is to be understood that instead of an RFID tag, the battery may include a serial number that can be conventionally read/detected using a hardware data line that is configured to be used with the battery through the wireless identification system 258. It is to be further understood that motor sensor/wireless identification system 262 is a conventional sensor and wireless identification system such as a tachometer and RFID tag that can be conventionally attached to or electronically connected to the motor 260, as will be discussed in greater detail later.

Another unique aspect of the present invention is the use of battery doors 254. Battery doors 254 are conventionally connected to protective casing 120 so that battery doors 254 can swing (or pivot) open so that battery 252 can be easily installed into power module 250 or removed from power module 250. In particular, the wearer 6 can remove battery 252 from power module 250 by opening battery doors 254 and removing battery 252 from power module 250. The battery 252 can then be placed on a conventional battery charger (not shown). Once the battery 252 has been fully charged, the wearer 6 can then remove the battery charger, open the battery doors 254, and slide the battery 252 into the power module 250 so that the battery 252 is securely retained within the power module 250. The wearer 6 then closes the battery doors 254 so that the battery 252 is not exposed to the elements. It is to be understood that a conventional locking mechanism 256 can be used to lock the battery 252 in place in the power module 250 so that the battery 252 does not inadvertently come loose while the ventilation system 100 is being operated.

### Yoke Module 300

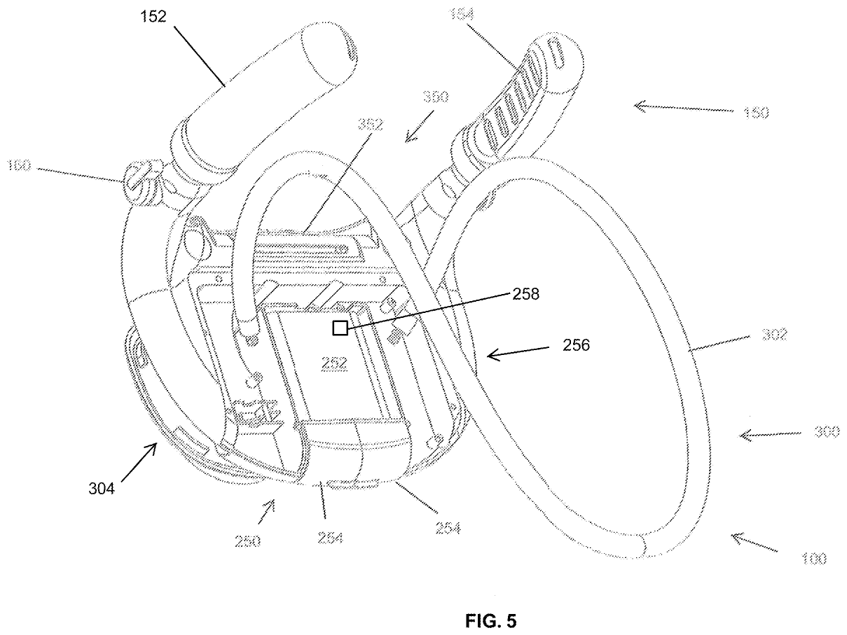

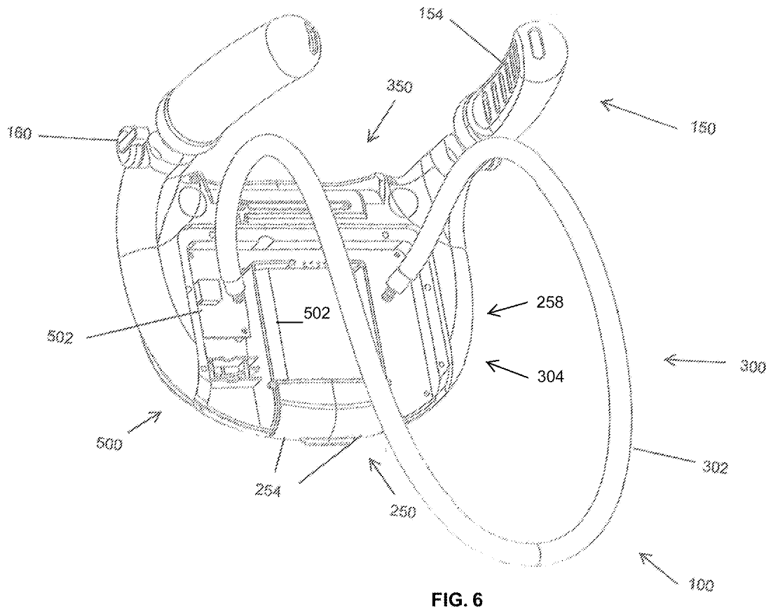

With respect to yoke module 300, as shown in FIGS. 5 and 6, yoke module 300, includes, in part, yoke 302 and yoke connectors 304. Preferably, yoke 302 is constructed of any suitable, durable, high strength, flexible, medical grade material. Preferably, yoke connectors 304 are attached to the back of protective casing 120.

Another unique aspect of the present invention is the use of yoke module 300. In particular, yoke module 300 can be used to assist in retaining ventilation system 100 on the shoulders of the wearer 6. Furthermore, yoke 302 is removably attached to protective casing through the use of yoke connectors 304. In this manner, yoke 302 can be easily attached to and removed from protective casing 120. Furthermore, since yoke 302 is flexible, yoke 302 can be adjusted so as to fit the upper torso of the wearer 6 so that ventilation system 100 will remain securely retained on the shoulders and the upper torso of the wearer 6. For example, the wearer 6 can position the ventilation system with the yoke module 300 installed over his/her head and place the yoke module 300 on the upper torso of the wearer 6 (FIG. 1). The wearer 6 can then pull/push on yoke 302 while yoke 302 is connected to yoke connectors 304 so that yoke 302 firmly contacts the upper torso of the wearer 6 in order to assist in retaining the ventilation system 100 on the shoulders and upper torso of the wearer 6.

### Neck Vent Module 350

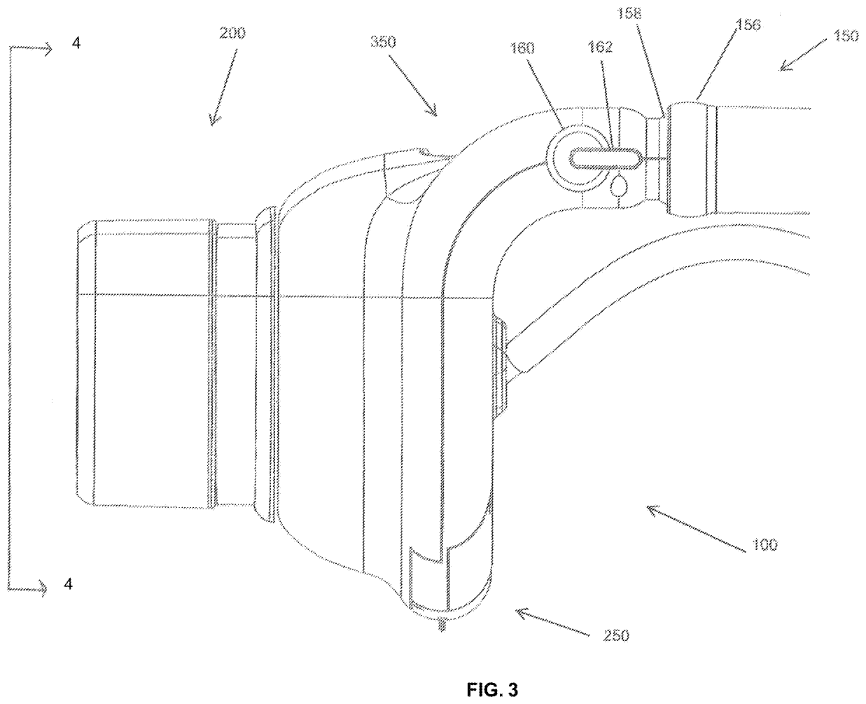

Regarding neck vent module 350, as shown in FIGS. 3-6, neck vent module 350, includes, in part, neck vent 352 (FIG. 4). Preferably, neck vent 352 is constructed of any suitable, durable, high strength, medical grade material.

### Air Flow Generation Module 450

Regarding air flow generation module 450, as shown in FIG. 4, air flow generation module 450, includes, in part, conventional electrical motor 260, conventional impeller 454, and back flow opening 456. It is to be understood that battery 252 provides the electrical power to electrical motor 260.

Another unique aspect of the present invention is the use of air flow generation module 450. In particular, as the electrical motor 260 causes the impeller 454 to rotate, the configuration of the impeller 454 causes air to be drawn through the air filter module 200. In this manner, the air filter module 200 can be used to filter the air being drawn into the ventilation system 100. Also, the back flow opening 456 is provided in order to allow air that is contained within the surgical hood 14 to also be drawn through back flow opening 456 in the direction of arrow D. In this manner, the back flow opening 456 provides for an even greater circulation of the air within the hood 14 while the ventilation system 100 is in operation.

### Printed Circuit Board (PCB) Module 500

With respect to printed circuit board (PCB) module 500, as shown in FIG. 6, printed circuit board module 500, includes, in part, a conventional printed circuit board 502. It is to be understood that printed circuit board 502 can be used to control the ventilation system 100 and interact with gown wireless identification system 30, air filtration module wireless identification system 208, power module wireless identification system 258, and motor sensor/wireless identification system 262, as will be described in greater detail later. In particular, printed circuit board 502 can be used to control the speed at which the impeller 454 (FIG. 4) rotates, thereby controlling the velocity of the air being emitted from the face vents 152 and the neck vent 352. It is to be further understood that the printed circuit board 502 is located in the rear of the protective casing 120 so that the printed circuit board can be located adjacent to the battery 252. Finally, it is to be understood that the printed circuit board 502 is conventionally retained within the protective casing 102 by conventional fasteners (not shown). Finally, it is to be understood that the printed circuit board (PCB) module 500 can utilize Bluetooth® low energy capabilities in order to allow the printed circuit board (PCB) module 500 to communicate with a mobile application that is conventionally installed on a remote computer 1565 (i.e., mobile communication device) such as a smartphone, tablet or data collection point.

### Operation of System for Managing Medical Device Maintenance and Medical Device Consumables

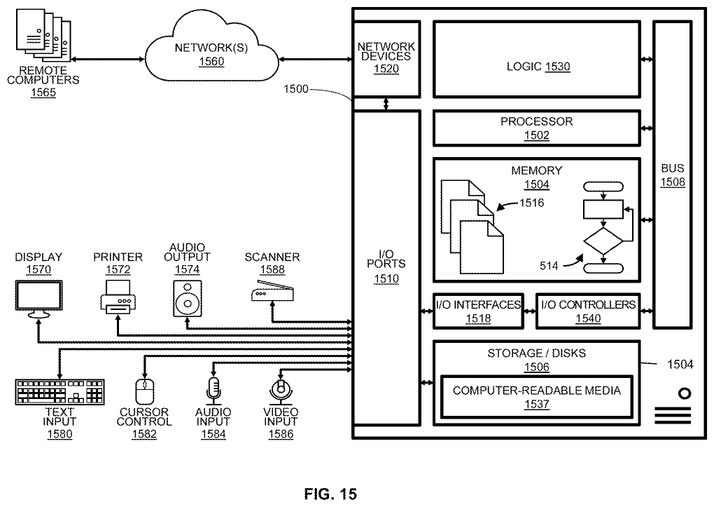

With respect to the operation of the system for managing medical device maintenance and medical device consumables, attention is directed to FIGS. 1-15. Assume that a medical device such as a surgical gown 4 (FIG. 1) is equipped with a conventional gown wireless identification system 30 having an RFID tag that can be conventionally attached to or electrically connected to the surgical gown 4, as discussed earlier. Secondly, assume that another medical device such as a fan filter 202 (FIG. 4) is equipped with a conventional wireless identification system 206 having an RFID tag that can be conventionally attached to or electronically connected to the air filter 202. Thirdly, assume that a still another medical device such as a battery 252 (FIGS. 4 and 5) is equipped with a conventional power module wireless identification system 258 having an RFID tag that can be conventionally attached to or electronically connected to the battery 252. Fourthly, assume that a fan motor 260 (FIG. 4) is equipped with a motor sensor/wireless identification system 262 having a tachometer 263 and RFID tag that can be conventionally attached to or electronically connected to the motor 260. It is to be understood that a processor 1502 (FIG. 15) can use the information from tachometer 263 or any other similar device in the motor sensor/wireless identification system 262 to determine the speed at which the motor 260 is operating and compare that speed to the speed at which the motor 260 should be preferably operating in order to determine if the filter 202 is clogged, the motor 206 is damaged, and/or the system 4 is otherwise malfunctioning to alert the user. Finally, assume that gown wireless identification system 30, air filtration module wireless identification system 208, power module wireless identification system 258, and motor sensor/wireless identification system 262 are in electrical communication with printed circuit board 502.

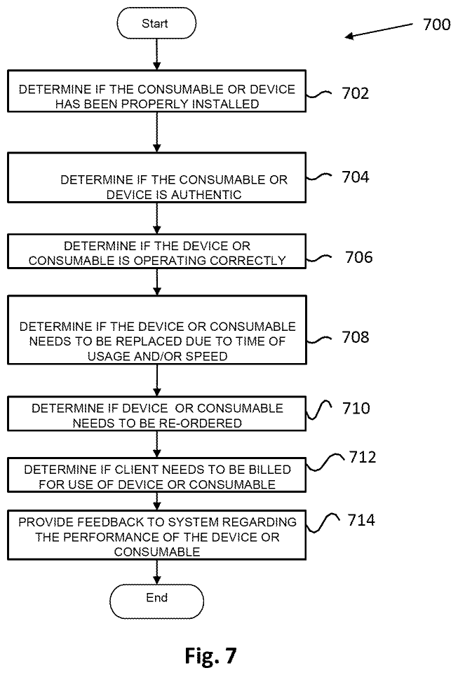

With reference to FIG. 7, a computer-implemented method 700 is illustrated that describes one embodiment for managing medical devices and medical device consumables. The method 700 is performed by at least a processor of a computer system that accesses and interacts with memories and/or data storage devices. For example, the processor at least accesses and reads/writes data to the memory and processes network communications to perform the actions of FIG. 7.

With continued reference to FIG. 7, at 702, the method 700 is initiated when a consumable such as a medical device (air filter 202 and/or a battery 252) is installed and a determination is made regarding whether or not the consumable is properly installed.

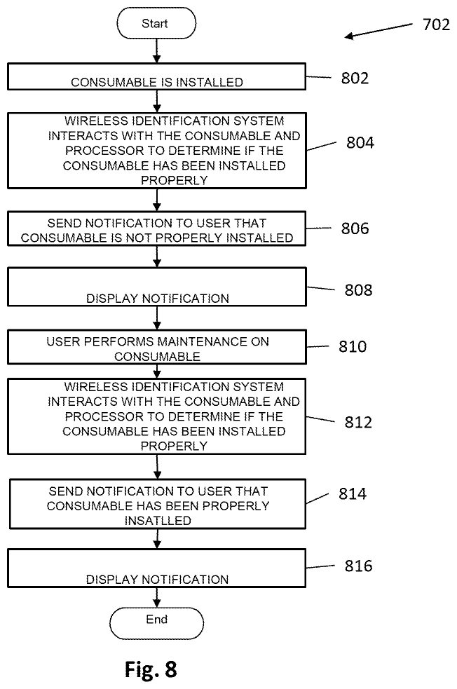

With respect to step 702, attention is now directed to FIG. 8. With reference to FIG. 8, a computer-implemented method 702 is illustrated that describes one embodiment for determining if the consumable or device has been properly installed. The method 702 is performed by at least a processor of a computer system that accesses and interacts with memories and/or data storage devices. For example, the processor at least accesses and reads/writes data to the memory and processes network communications to perform the actions of FIG. 8.

With reference to FIG. 8, at 802, the method 702 is initiated when a consumable such as a medical device (air filter 202 and/or a battery 252) is installed. For the purposes of this example, assume that the consumable such as battery 252 is installed in the wrong orientation. This is a unique aspect of the present invention because if the consumable is not properly installed, this may cause the helmetless support system 2 for use with surgical hoods and gowns to not operate or not operate correctly.

In block 804, in one embodiment, the power module wireless identification system 258 will conventionally sense that the battery 252 has been installed in the wrong orientation and send a signal to the processor 1502. For example, in one embodiment, the power module wireless identification system 258 will conventionally interact with an RFID tag 253 on the battery 252 to determine that the battery 252 is not installed in the correct orientation.

In block 806, the processor 1502 can then send a notification through the display 1570 (FIG. 15) to the user that the consumable such as battery 252 has not been installed properly.

In block 808, the notification can then be viewed by the user on a display 1570 such as a smartphone display to inform the user that the consumable has been installed incorrectly. It is to be understood that processor 1502 can also send an alert such as an audible and haptic (touch) alarm to the user to alert the user that the consumable such as battery 252 has not been installed properly.

In block 810, the user performs maintenance on the consumable, in one embodiment, by correcting the orientation of the consumable.

In block 812, the power module wireless identification system 258 will conventionally again interact with an RFID tag 253 on the battery 252 to determine that the battery 252 is now installed correctly, for example, the battery 252 is installed in the correct orientation.

In block 814, the processor 1502 can then send a notification to the user that the consumable such as battery 252 has been installed properly. It is to be understood that along with sending a notification to the user, in one embodiment, the processor 1502 can 1.) conventionally prevent the helmetless support system 2 for use with surgical hoods and gowns from being turned on, 2.) send a signal to the audio output 1575 to emit a beep or other similar sound or a spoken voice notification to notify the user, 3.) notify a remote computer 1565 associated with a system administrator to send warning alerts to the medical personnel tasked with maintaining the helmetless support system 2, or the like. An important consideration being that the user and/or the personnel tasked with maintaining the helmetless support system 2 are provided with a notification or alert that there is an issue with the consumable.

In block 816, the notification can then be viewed by the user on a display 1570 such as a smartphone display to inform the user that the consumable has been installed correctly. It is to be understood that processor 1502 can also send an alert such as an audible and haptic (touch) alarm to the user to alert the user that the consumable such as battery 252 has been installed properly.

It is to be understood that while the method 702, as set forth in FIG. 8, is described in terms of the correct installation of battery 252, the method 702 can also be applied to the correct installation of filter 202, gown 4, motor 260, etc.

With continued reference to FIG. 7, after it has been determined that the consumable has been installed correctly, at block 704, a determination is made as to whether or not the consumable is authentic. This is a unique aspect of the present invention because the use a consumable that is not authentic may cause the helmetless support system 2 for use with surgical hoods and gowns to operate correctly.

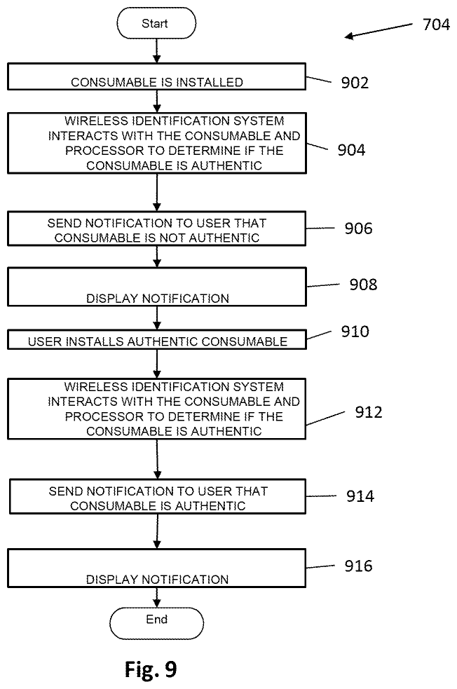

With respect to step 704, attention now is directed to FIG. 9. With reference to FIG. 9, a computer-implemented method 704 is illustrated that describes one embodiment for determining if the consumable or device is authentic. The method 704 is performed by at least a processor of a computer system that accesses and interacts with memories and/or data storage devices. For example, the processor at least accesses and reads/writes data to the memory and processes network communications to perform the actions of FIG. 9.

With reference to FIG. 9, at 902, the method 704 is initiated when a consumable such as a medical device (air filter 202 and/or a battery 252) is installed. For the purposes of this example, assume that the consumable such as battery 252 is not an authentic battery in that the battery 252 may not be a battery that is recommended for use by the helmetless support system 2 for use with surgical hoods and gowns.

In block 904, the power module wireless identification system 258 will conventionally sense that the battery 252 is not authentic and send a signal to the processor 1502. For example, in one embodiment, the power module wireless identification system 258 will conventionally interact with an RFID tag 253 on the battery 252 to determine that the battery 252 is not authentic. In particular, the power module wireless identification system 258 may conventionally interact with data storage 1506 to determine if the information from RFID tag 253 associated with battery 252 matches RFID tag information associated with authentic batteries stored in data storage 1506. Furthermore, preferably, the RFID tag 253 should be conventionally encrypted so that a determination can be made as to the authenticity of the RFID tag 253 or that that particular RFID tag 253 has been used before.

It is to be understood that system may not be able to determine if a consumable is not authentic or if the consumable is used up, as will be described in greater detail later. This is because the system may not have the full history of the consumable and may have limited available storage. However, the system can report what data the system measures to an application associated with a remote device 1565 that is associated with the user or the system administrator and/or the system can conventionally transmit the data to a cloud computing system, which can combine that with other consumable history to determine if the consumable is “used up”. In this manner, the system can send a message back down from the cloud computing system to the app to the remote device 1565 associated with the user or the system administrator to conventionally configure the remote device 1565 to emit a sound such as a beep (or a spoken voice alert) or even configure the helmetless support system 2 to not operate depending on if all of the consumables on the helmetless support system 2 are properly connected. An important consideration being that all of the consumables on the helmetless support system 2 need to work if there is no app installed on the remote device 1565 and do a store and forward approach with the data from the consumables. In short, in some cases these notifications may not be “real time,”

It is to be further understood that in another embodiment, the power module wireless identification system 258 could be configured to run conventional intelligence (Al) algorithms to determine if the consumable is authentic or counterfeit based upon sampling or machine learning. In particular, counterfeit devices could have valid RFID tags in stored in the system, but if the same RFID is used at multiple locations, for example, the system would be able to eventually catch this in the cloud computing system.

In block 906, if it is determined that the consumable is not authentic, the processor 1502 can then send a notification to the user that the consumable such as battery 252 is not authentic.

In block 908, the notification can then be viewed by the user on a display 1570 such as a smartphone display to inform the user that the consumable is not authentic. It is to be understood that processor 1502 can also send an alert such as an audible and haptic (touch) alarm to the user to alert the user that the consumable such as battery 252 is not authentic.

In block 910, the user removes the consumable that is not authentic and installs an authentic consumable.

In block 912, the power module wireless identification system 258 will conventionally again interact with an RFID tag 253 on the newly installed battery 252 to determine that the consumable (battery 252) that is now installed is authentic.

In block 914, the processor 1502 can then send a notification to the user that the consumable such as battery 252 is authentic.

In block 916, the notification can then be viewed by the user on a display 1570 such as a smartphone display to inform the user that the consumable that is now installed is authentic. It is to be understood that processor 1502 can also send an alert such as an audible and haptic (touch) alarm to the user to alert the user that the newly installed consumable, such as battery 252, is authentic.

It is to be understood that while the method 704, as set forth in FIG. 9, is described in terms of the authenticity of battery 252, the method 704 can also be applied to the authenticity of filter 202, gown 4, motor 260, etc.

With continued reference to FIG. 7, after it has been determined that the consumable is authentic, at block 706, a determination is made as to whether or not the consumable or device is operating correctly. This is a unique aspect of the present invention because if the consumable is not operating correctly, this may cause the helmetless support system 2 for use with surgical hoods and gowns to overheat, malfunction, perform poorly, or the like.

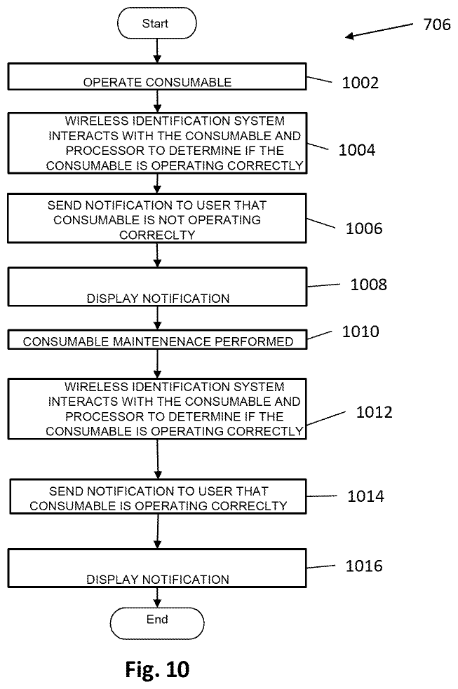

With respect to step 706, attention is directed to FIG. 10. With reference to FIG. 10, a computer-implemented method 706 is illustrated that describes one embodiment for determining if the consumable or device is operating correctly. The method 706 is performed by at least a processor of a computer system that accesses and interacts with memories and/or data storage devices. For example, the processor at least accesses and reads/writes data to the memory and processes network communications to perform the actions of FIG. 10.

With reference to FIG. 10, at 1002, the method 704 is initiated when helmetless support system 2 for use with surgical hoods and gowns is activated and a consumable such as a medical device (air filter 202 and/or a battery 252) is operating. For the purposes of this example, assume that the consumable, such as battery 252, is not operating correctly and is overheating.

In block 1004, the power module wireless identification system 258 will conventionally sense that the battery 252 is not operating correctly and is overheating. For example, in one embodiment, the power module wireless identification system 258 will conventionally interact with a conventional sensor 255 (FIG. 4) on the battery 252 to determine that the battery 252 is not operating correctly and is overheating.

In block 1006, if it is determined that the consumable is not operating correctly and is overheating, the processor 1502 can then send a notification to the user that the consumable such as battery 252 is not operating correctly and is overheating.

In block 1008, the notification can then be viewed by the user on a display 1570 such as a smartphone display to inform the user that the consumable is not operating correctly and is overheating. It is to be understood that processor 1502 can also send an alert such as an audible and haptic (touch) alarm to the user to alert the user that the consumable such as battery 252 is not operating correctly.

In block 1010, maintenance is performed on the consumable such as battery 252. For example, the malfunctioning battery 252 may be removed from the power module 250 and a new battery 252 may then be inserted into the power module 250.

In block 1012, the power module wireless identification system 258 will conventionally again interact with the sensor 255 on the newly installed battery 252 to determine that the newly installed consumable (battery 252) is now operating correctly.

In block 1014, the processor 1502 can then send a notification to the user that the consumable such as battery 252 is now operating correctly.

In block 1016, the notification can then be viewed by the user on a display 1570 such as a smartphone display to inform the user that the consumable that is now operating correctly. It is to be understood that processor 1502 can also send an alert such as an audible and haptic (touch) alarm to the user to alert the user that the consumable such as battery 252 is now operating correctly.

It is to be understood that while the method 706, as set forth in FIG. 10, is described in terms of the operation of battery 252, the method 706 can also be applied to the operation/performance characteristics of filter 202, gown 4, fan motor 260, etc.

With continued reference to FIG. 7, after it has been determined that the consumable is operating correctly, at block 708, a determination is made as to whether or not the consumable needs to be replaced such as for exceeding a time of usage and speed (or other similar factors of related to usage). This is a unique aspect of the present invention because if the consumable has expired or it has exceeded its time of usage, this may cause the helmetless support system 2 for use with surgical hoods and gowns to not operate correctly, for example, the fan motor 260 may not be operating at a desired speed.

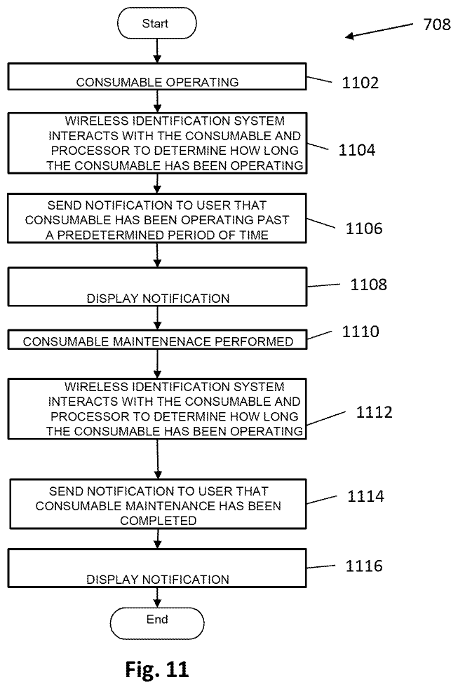

With respect to step 708, attention is directed to FIG. 11. With reference to FIG. 11, a computer-implemented method 708 is illustrated that describes one embodiment for determining whether or not the consumable needs to be replaced such as for exceeding a time of usage. The method 708 is performed by at least a processor of a computer system that accesses and interacts with memories and/or data storage devices. For example, the processor at least accesses and reads/writes data to the memory and processes network communications to perform the actions of FIG. 11.

With reference to FIG. 11, at 1102, the method 708 is initiated when helmetless support system 2 for use with surgical hoods and gowns has been activated and a consumable such as a medical device (air filter 202 and/or a battery 252) is operating. For the purposes of this example, assume that the consumable, such as battery 252, has been operating for a period of time.

In block 1104, the power module wireless identification system 258 will conventionally sense that the battery 252 has been operating for a period of time. For example, in one embodiment, the power module wireless identification system 258 will conventionally interact with a conventional sensor 255 (FIG. 4) on the battery 252 to determine that the battery 252 has been operating for a period of eight (8) hours. Furthermore, the power module wireless identification system 258 interacts with processor 1502 and storage 1506 to determine that the battery 252 should not operate for more than ten (10) hours, for example. If an upcoming surgical procedure is estimated to take three (3) hours and the recommended operating time for the battery 252 is ten (10) hours, the battery 252 may need to be replaced prior to the upcoming medical procedure to ensure that the battery 252 will have enough power to last through the upcoming surgical procedure.

In block 1106, if it is determined that the consumable will be operating for more than a predetermined period of time and the consumable needs to be replaced, the processor 1502 can then send a notification through the power module wireless identification system 258 to the user that the consumable, such as battery 252, needs to be replaced since it will be operating past the recommended time of usage of the battery 252, if used during the upcoming medical procedure.

In block 1108, the notification can then be viewed by the user on a display 1570 such as a smartphone display to inform the user that the consumable such as battery 252 needs to be replaced since it will be operating past the recommended time of usage of the battery 252, if used during the upcoming medical procedure. It is to be understood that processor 1502 can also send an alert such as an audible and haptic (touch) alarm to the user to alert the user that the consumable such as battery 252 needs to be replaced since it will be operating past the recommended time of usage of the battery 252, if used during the upcoming medical procedure.

In block 1110, maintenance is performed on the consumable such as battery 252. For example, battery 252 may be removed from the power module 250 and a new battery 252 may then be inserted into the power module 250.

In block 1112, the power module wireless identification system 258 will conventionally again interact with the sensor 255 on the newly installed battery 252 to determine that the newly installed consumable (battery 252) has been operating less than the predetermined period of time and will have enough power to operate during the upcoming medical procedure.

In block 1114, the processor 502 can then send a notification through the power module wireless identification system 258 to the user that the maintenance on the consumable such as battery 252 has been completed.

In block 1116, the notification can then be viewed by the user on a display 1570 such as a smartphone display to inform the user that the maintenance on the consumable such as battery 252 has been completed. It is to be understood that processor 1502 can also send an alert such as an audible and haptic (touch) alarm to the user to alert the user that the maintenance on the consumable such as battery 252 has been completed.

It is to be understood that while the method 708, as set forth in FIG. 11, is described in terms of the operation of battery 252, the method 708 can also be applied to the operation/performance characteristics of filter 202, gown 4, fan motor 260, etc. For example, filter 202 can be checked to make sure filter 202 still has usable life on it (based on operating time and fan speed during those times). Furthermore, the gown 4 can be checked to make sure it has been replaced for the upcoming surgical procedure.

With continued reference to FIG. 7, after it has been determined if the consumable needs to be replaced, at block 710, a determination is made as to whether or not the consumable needs to be re-ordered. This is a unique aspect of the present invention because if the consumable has been replaced and the consumable needs to be re-ordered, the present invention will keep track of the inventory of the consumables being used at the hospital or medical practice and ensure that the hospital or medical practice has a sufficient amount of the consumables on hand for subsequent medical procedures.

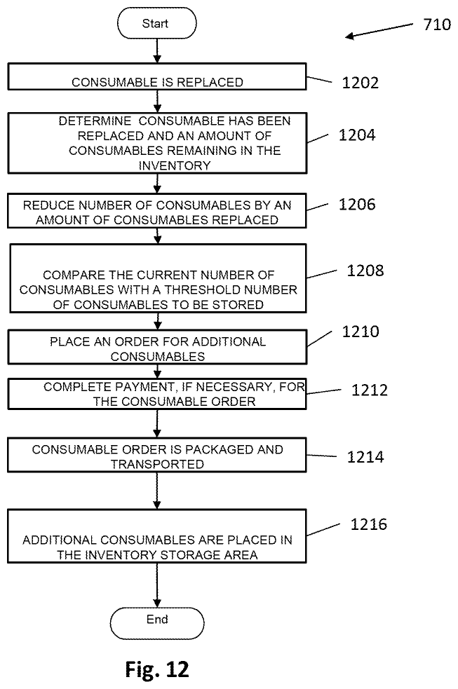

With respect to step 710, attention now is directed to FIG. 12. With reference to FIG. 12, a computer-implemented method 710 is illustrated that describes one embodiment for determining whether or not the consumable needs to be re-ordered. The method 710 is performed by at least a processor of a computer system that accesses and interacts with memories and/or data storage devices. For example, the processor at least accesses and reads/writes data to the memory and processes network communications to perform the actions of FIG. 12.

With reference to FIG. 12, at block 1202, the method 710 is initiated when a consumable for helmetless support system 2 has been replaced, as discussed above with respect to step 708 and FIG. 11. For the purposes of this example, assume that the consumable such as battery 252 has been replaced and a new inventory of batteries needs to be re-ordered.

In block 1204, the power module wireless identification system 258 will conventionally sense that the consumable (battery 252) has been replaced, as discussed above, and power module wireless identification system 258 will interact with processor 1502 to determine the amount of the consumable (batteries 252) that are remaining in the consumable (battery) inventory. Typically, a threshold number of consumables (batteries 252) that are to be stored in a physical inventory (such as a consumable storeroom (not shown) of the consumables (batteries) will be maintained on data storage 1506.

In block 1206, when a consumable (battery 252) is replaced and a new consumable (battery 252) is taken from the physical inventory, the number of consumables (batteries 252) in the data storage 1506 will be reduced by the amount of the consumables replaced, in this case, one (1).

In block 1208, the power module wireless identification system 258 will conventionally compare the current number of consumables (batteries 252) in the physical storage, as stored in data storage 1506, with a threshold number of consumables (batteries 252) that are desired to be stored in the physical inventory of consumables (batteries 252), as stored in data storage 1506.

In block 1210, if the current number of consumables (batteries 252) is (or will be) below the threshold number of consumables (batteries 252) that are desired to be stored in the physical inventory of consumables (batteries 252), in one embodiment, processor 1506 can conventionally place an order for an additional number of consumables (batteries 252) with a desired supplier. In another embodiment, power module wireless identification system 258 can send a notification to the user that additional consumables (batteries 252) need to be ordered. In this manner, the user can view the notification on the display 1570 (FIG. 5) so that the user will be able to take subsequent action with respect to ordering additional consumables.

In block 1212, payment, if necessary, is completed for the consumable order.

In block 1214, after the payment for the consumable order has been completed, the additional consumables (batteries 252) that have been ordered are packaged and transported to the medical provider and/or hospital. In particular, in one embodiment, the processor 1502 transmits a message (which includes the order) to a remote computer 1565. In one embodiment, the remote computer 1565 may be associated with a medical supplier's warehouse (or fulfillment center) or a sales channel (retailer's store). In another embodiment, the message may contain an order for an amount of inventory of the consumable to be ordered.

After the message is received at the remote computer 1565, the order is fulfilled by retrieving the ordered consumables. If the order is sent to one of the supplier's warehouses (or fulfillment center), warehouse-management software receives the order, and systems at the warehouse fulfill the order by retrieving the units of the ordered consumables from a unit storage location, packaging the ordered consumables, and preparing them for shipping. If the order is sent to the sales channel (supplier's store), the order is fulfilled at the store. In one embodiment, the systems are controlled at least in part by the instructions in the order. The systems may include, for example, automated robotic machines or mechanisms configured to locate and retrieve the ordered consumables from warehouse or store locations based on the order and/or rearrange the ordered consumables based on the order (e.g., add selected ordered consumables, remove selected ordered consumables, based on the final ordered consumables replenishment policy). The retrieved ordered consumables are delivered by the robotic mechanism to automated packaging mechanisms that package the retrieved ordered consumables in the warehouse.

In block 1214, if the retrieved ordered consumables are located at the warehouse, the retrieved ordered consumables are transported to a sales channel (supplier's store).

In block 1216, in another embodiment, if the retrieved ordered consumables are at the supplier's store, the retrieved ordered consumables are delivered to the user's physical inventory storage area and placed in physical inventory storage area for subsequent use by the user.

In one embodiment, the robotic mechanisms may include one or more robots configured to navigate throughout a given warehouse or store, locate and retrieve items, and carry items to a destination. Each robot may include at least a body structure, a power source, a control interface, a wired/wireless communication interface, a drive device to move the robot, a navigation device, one or more sensors, and/or a balance device. Of course, the robot may be configured in different ways and multiple different types of robots may be operating together in the robotic mechanism of the warehouse or store.

In one or more embodiments, the robotic mechanisms may include one or more of the following systems. Automated Guided Vehicles (AGVs) for transporting materials, supplies, and inventory within warehouse or store facilities. An AGV may be configured to autonomously navigate warehouse or store facilities by following defined routes marked by wires, magnetic strips, tracks, sensors embedded in the floor or other physical guides. The AGV may also be navigated by a defined map of the warehouse or store based on a coordinate system and a tracked location of the AGV. Cameras may also be used to navigate an AGV.

Another robotic mechanism is Automated Storage and Retrieval Systems (AS/RS) which may include a group of computer-controlled systems that automate inventory management and store/retrieve goods on demand from storage locations in the warehouse or store. The AS/RS may operate either as cranes or shuttles on fixed tracks and can traverse product aisles and vertical heights to remove items or drop off items from storage. Another robotic mechanism is articulated robotic arms that are a type of pick-and-place robot. These arms can move, turn, grab/release, lift and move items with, for example, multi-jointed limbs used to manipulate products.

After the retrieved ordered consumable is transported to a packaging area of the warehouse, the retrieved ordered consumable for the order are packaged by an automated system. This may include cartonization software that determines quantity, size, and type of container required to package the order, and/or bagging machines that help speed up packaging operations. As discussed above, after the retrieved ordered consumable are transported to the physical inventory storage area, the retrieved ordered consumable can be placed on a designated area of the physical inventory storage area store.

The packaged order is transferred to a shipper, or if the supplier has its own shipment operation, then the supplier's own trucks will take the shipment. The status of the order is changed to “in transit” in the inventory-management system. The order arrives at the user's consumable storage location it is destined for. Finally, the computerized management system software then updates the inventory position of the retrieved ordered consumable at the user's consumable storage location.

On the other hand, if the order is sent to a manufacturer, then the sequence of steps is similar except that the manufacturer is responsible for acting as a fulfillment center and fulfilling the order.

It is to be understood that while the method 710, as set forth in FIG. 12, is described in terms of the operation of battery 252, the method 710 can also be applied to the operation/performance characteristics of filter 202, gown 4, fan motor 260, etc. For example, filter 202 can be re-ordered when the number of filters 202 in the filter inventory falls below a threshold number of filters 202 that are desired to be on-hand in the filter storage area (not shown). Furthermore, gowns 4 can be re-ordered when the number of gowns 4 in the gown inventory falls below a threshold number of gowns 4 that are desired to be on-hand in the gown storage area (not shown).

With continued reference to FIG. 7, after it has been determined if the consumable needs to be re-ordered, at block 712, a determination is made as to whether or not the client needs to be billed for the use of the device and/or consumable. This is a unique aspect of the present invention because if the consumable has been replaced and the consumable needs to be re-ordered, the present invention will determine if the client needs to be billed for the use of the device and/or consumable. For example, an order may be placed at a negotiated rate/term that is stored in the data storage 1506 and then conventionally invoice or bill the hospital or medical practice according to those negotiated rates/terms.

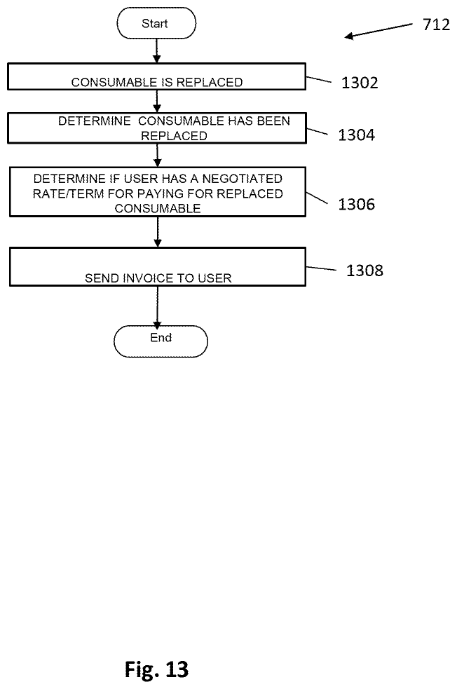

With respect to step 712, attention is directed to FIG. 13. With reference to FIG. 13, a computer-implemented method 712 is illustrated that describes one embodiment for determining whether or not the client needs to be billed for the use of the device and/or consumable. The method 712 is performed by at least a processor of a computer system that accesses and interacts with memories and/or data storage devices. For example, the processor at least accesses and reads/writes data to the memory and processes network communications to perform the actions of FIG. 13.

With reference to FIG. 13, at 1302, the method 712 is initiated when a consumable for helmetless support system 2 has been replaced, as discussed above with respect to step 708 and FIG. 11. For the purposes of this example, assume that the consumable such as battery 252 has been replaced and a new inventory of batteries needs to be re-ordered.

In block 1304, the power module wireless identification system 258 will conventionally sense that the consumable (battery 252) has been replaced, as discussed above. For example, in one embodiment, the power module wireless identification system 258 will conventionally interact with an RFID tag 253 on the battery 252 to determine that the battery 252 has been replaced.

In block 1306, when a consumable (battery 252) is replaced, and power module wireless identification system 258 will conventionally interact with processor 1502 and data storage 1506 to determine if the user of the consumable (battery 252) has a negotiated rate/term of payment for the replaced consumable (battery 252).

In block 1308, the power module wireless identification system 258 will then interact with processor 502 and network 1560 to transmit an invoice to the user of the consumable (battery 252) through a remote computer 1565 associated with the user. In one embodiment, if the user does have a negotiated rate/term of payment for the consumable, the user will be notified that the consumable (battery 252) has been replaced and that a new consumable (battery 252) has been taken out of inventory. The user's account will then be charged for the new consumable (battery 252). In another embodiment, if the user does not have a negotiated rate/term of payment, an invoice for payment of the new consumable (battery 252) will be conventionally prepared and sent to the user.

It is to be understood that while the method 712, as set forth in FIG. 13, is described in terms of the operation of battery 252, the method 712 can also be applied to the replacing, re-ordering, and billing of filter 202, gown 4, fan motor 260, etc. For example, filter 202 can be re-ordered and billed when the number of filters 202 in the filter inventory falls below a threshold number of filters 202 that are desired to be on-hand in the filter storage area (not shown). Furthermore, gowns 4 can be re-ordered and billed when the number of gowns 4 in the gown inventory falls below a threshold number of gowns 4 that are desired to be on-hand in the gown storage area (not shown). Finally, fan motor 260 can be re-ordered and billed when the number of fan motors 260 in the fan motor inventory falls below a threshold number of fan motor 260 that are desired to be on-hand in the fan motor storage area (not shown).

With continued reference to FIG. 7, at block 714, helmetless support system 2 can also be used to provide feedback regarding the performance of the device or consumable. This is a unique aspect of the present invention because if the user of helmetless support system 2 can receive information regarding the performance of the various devices and consumables associated with helmetless support system 2, then the user will be able to more easily and quickly determine if there are any operational issues with helmetless support system 2, and correct those operational issues before the user employs helmetless support system 2 in a medical procedure or while the user employs helmetless support system 2 during a medical procedure.

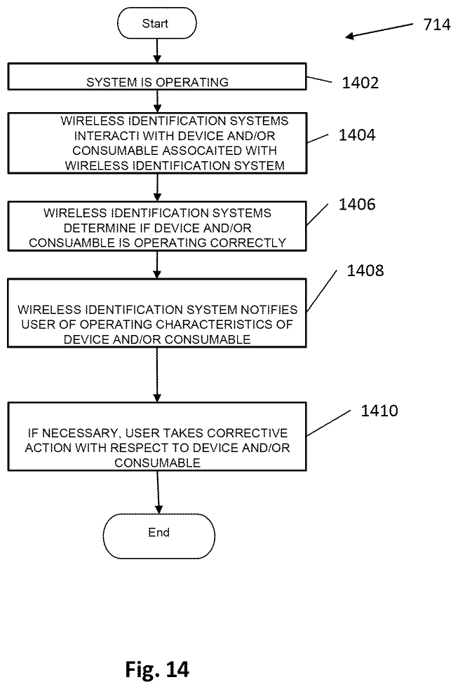

With respect to step 714, attention is directed to FIG. 14. With reference to FIG. 14, a computer-implemented method 714 is illustrated that describes one embodiment for providing feedback regarding the performance of the device or consumable. The method 714 is performed by at least a processor of a computer system that accesses and interacts with memories and/or data storage devices. For example, the processor at least accesses and reads/writes data to the memory and processes network communications to perform the actions of FIG. 14.

With reference to FIG. 14, at 1402, the method 714 is initiated when helmetless support system 2 is put into operation, as discussed above. For the purposes of this example, assume that the consumable such as battery 252 is being monitored by power module wireless identification system 258.

In block 1404, the power module wireless identification system 258 will conventionally interact with sensor 255 on consumable (battery 252) to determine the operating characteristics of the consumable (battery 252). For example, the amount of battery life in battery 252 can be conventionally measured with sensor 255. The voltage or battery 252 can be conventionally measured with sensor 255. The amount of time that battery 252 has been operating can be conventionally measured with sensor 255. The amount of battery life in battery 252 can be conventionally measured with sensor 255. An important consideration being that the operational characteristics of the consumable (battery 252) can be determined so that the user (or service technician) can determine if the consumable and/or device is operating correctly. Also, this information can be used by the user (or service technician) to determine if the device and/or consumable will be able to continue operating correctly during a medical procedure, especially a lengthy medical procedure.

In block 1406, the power module wireless identification system 258 will determine if the device and/or consumable (battery 252) is operating based upon the measured device and/or consumable operating characteristics, as discussed above.

In block 1408, the power module wireless identification system 258 can send a notification to the user (or service technician) through a display 1570 (FIG. 15) associated with a remote computing device 1565 which is associated with the user (or technician). In one embodiment, the notification can include, but not limited to, the various operating characteristics of the device and/or consumable, an audible or haptic (touch) warning or alert that a particular device and/or consumable is not operating correctly, and a recommendation for the user (or service technician) regarding how to correct any issues with the device and/or consumable that is not operating correctly.

In block 1410, the user (or service technician) can perform the recommended corrective action on the device and/or consumable based upon the notification.

It is to be understood that while the method 714, as set forth in FIG. 14, is described in terms of the operation of battery 252, the method 714 can also be applied to the operation of filter 202, fan motor 260, etc. For example, the operation of filter 202 can be monitored by air filtration module wireless identification system 208. Also, fan motor 260 can be monitored by motor sensor/wireless identification system 262.

Another unique aspect of the present invention is that the feedback regarding the performance of the device or consumable can be used with a machine learning model or an artificial intelligence model to forecast performance characteristics of the devices and/or consumables associated with the helmetless support system 2 and provide recommendations as to improving the operating characteristics of the devices and/or consumables associated with the helmetless support system 2.

### Computing Device Embodiment

FIG. 15 illustrates an example computing device that is configured and/or programmed as a special purpose computing device with one or more of the example systems and methods described herein, and/or equivalents. The example computing device may be a computer 1500 that includes at least one hardware processor 1502, a memory 1504, and input/output ports 1510 operably connected by a bus 1508. In one example, the computer 1500 may include logic 1530 similar to logic/systems 702, 704, 706, 708, 710, 712, and 714, and 400 shown in FIGS. 7-14.

In different examples, the logic 1530 may be implemented in hardware, a non-transitory computer-readable medium 1537 with stored instructions, firmware, and/or combinations thereof. While the logic 1530 is illustrated as a hardware component attached to the bus 1508, it is to be appreciated that in other embodiments, the logic 1530 could be implemented in the processor 1502, stored in memory 1504, or stored in disk 1506.

In one embodiment, logic 1530 or the computer is a means (e.g., structure: hardware, non-transitory computer-readable medium, firmware) for performing the actions described. In some embodiments, the computing device may be a server operating in a cloud computing system, a server configured in a Software as a Service (SaaS) architecture, a smart phone, laptop, tablet computing device, and so on.

The means may be implemented, for example, as an ASIC programmed to predict a product demand. The means may also be implemented as stored computer executable instructions that are presented to computer 1500 as data 1516 that are temporarily stored in memory 1504 and then executed by processor 1502.

Logic 1530 may also provide means (e.g., hardware, non-transitory computer-readable medium that stores executable instructions, firmware) for predicting a product demand.

Generally describing an example configuration of the computer 1500, the processor 1502 may be a variety of various processors including dual microprocessor and other multi-processor architectures. A memory 1504 may include volatile memory and/or non-volatile memory. Non-volatile memory may include, for example, ROM, PROM, and so on. Volatile memory may include, for example, RAM, SRAM, DRAM, and so on.

A storage disk 1506 may be operably connected to the computer 1500 via, for example, an input/output (I/O) interface (e.g., card, device) 1518 and an input/output port 1510 that are controlled by at least an input/output (I/O) controller 1540. The disk 1506 may be, for example, a magnetic disk drive, a solid-state disk drive, a floppy disk drive, a tape drive, a Zip drive, a flash memory card, a memory stick, and so on. Furthermore, the disk 1506 may be a CD-ROM drive, a CD-R drive, a CD-RW drive, a DVD ROM, and so on. The memory 1504 can store a process 1514 and/or a data 1516, for example. The disk 1506 and/or the memory 1504 can store an operating system that controls and allocates resources of the computer 1500.

The computer 1500 may interact with, control, and/or be controlled by input/output (I/O) devices via the input/output (I/O) controller 1540, the I/O interfaces 1518, and the input/output ports 1510. Input/output devices may include, for example, one or more displays 1570, printers 1572 (such as inkjet, laser, or 3D printers), audio output devices 1574 (such as speakers or headphones), text input devices 1580 (such as keyboards), cursor control devices 1582 for pointing and selection inputs (such as mice, trackballs, touch screens, joysticks, pointing sticks, electronic styluses, electronic pen tablets), audio input devices 1584 (such as microphones or external audio players), video input devices 1586 (such as video and still cameras, or external video players), image scanners 1588, video cards (not shown), disks 1506, network devices 1520, and so on. The input/output ports 1510 may include, for example, serial ports, parallel ports, and USB ports.

The computer 1500 can operate in a network environment and thus may be connected to the network devices 1520 via the I/O interfaces 1518, and/or the I/O ports 1510. Through the network devices 1520, the computer 1500 may interact with a network 1560. Through the network, the computer 500 may be logically connected to remote computers 1565. Networks with which the computer 1500 may interact include, but are not limited to, a LAN, a WAN, and other networks.

Definitions and Other Embodiments

In another embodiment, the described methods and/or their equivalents may be implemented with computer executable instructions. Thus, in one embodiment, a non-transitory computer readable/storage medium is configured with stored computer executable instructions of an algorithm/executable application that when executed by a machine(s) cause the machine(s) (and/or associated components) to perform the method. Example machines include but are not limited to a processor, a computer, a server operating in a cloud computing system, a server configured in a Software as a Service (SaaS) architecture, a smart phone, and so on). In one embodiment, a computing device is implemented with one or more executable algorithms that are configured to perform any of the disclosed methods.

In one or more embodiments, the disclosed methods or their equivalents are performed by either: computer hardware configured to perform the method; or computer instructions embodied in a module stored in a non-transitory computer-readable medium where the instructions are configured as an executable algorithm configured to perform the method when executed by at least a processor of a computing device.

While for purposes of simplicity of explanation, the illustrated methodologies in the figures are shown and described as a series of blocks of an algorithm, it is to be appreciated that the methodologies are not limited by the order of the blocks. Some blocks can occur in different orders and/or concurrently with other blocks from that shown and described. Moreover, less than all the illustrated blocks may be used to implement an example methodology. Blocks may be combined or separated into multiple actions/components. Furthermore, additional and/or alternative methodologies can employ additional actions that are not illustrated in blocks. The methods described herein are limited to statutory subject matter under 35 U.S.C § 101.

The following includes definitions of selected terms employed herein. The definitions include various examples and/or forms of components that fall within the scope of a term and that may be used for implementation. The examples are not intended to be limiting. Both singular and plural forms of terms may be within the definitions.

References to “one embodiment”, “an embodiment”, “one example”, “an example”, and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

A “data structure”, as used herein, is an organization of data in a computing system that is stored in a memory, a storage device, or other computerized system. A data structure may be any one of, for example, a data field, a data file, a data array, a data record, a database, a data table, a graph, a tree, a linked list, and so on. A data structure may be formed from and contain many other data structures (e.g., a database includes many data records). Other examples of data structures are possible as well, in accordance with other embodiments.

“Computer-readable medium” or “computer storage medium”, as used herein, refers to a non-transitory medium that stores instructions and/or data configured to perform one or more of the disclosed functions when executed. Data may function as instructions in some embodiments. A computer-readable medium may take forms, including, but not limited to, non-volatile media, and volatile media. Non-volatile media may include, for example, optical disks, magnetic disks, and so on. Volatile media may include, for example, semiconductor memories, dynamic memory, and so on. Common forms of a computer-readable medium may include, but are not limited to, a floppy disk, a flexible disk, a hard disk, a magnetic tape, other magnetic medium, an application specific integrated circuit (ASIC), a programmable logic device, a compact disk (CD), other optical medium, a random access memory (RAM), a read only memory (ROM), a memory chip or card, a memory stick, solid state storage device (SSD), flash drive, and other media from which a computer, a processor or other electronic device can function with. Each type of media, if selected for implementation in one embodiment, may include stored instructions of an algorithm configured to perform one or more of the disclosed and/or claimed functions. Computer-readable media described herein are limited to statutory subject matter under 35 U.S.C § 101.

“Logic”, as used herein, represents a component that is implemented with computer or electrical hardware, a non-transitory medium with stored instructions of an executable application or program module, and/or combinations of these to perform any of the functions or actions as disclosed herein, and/or to cause a function or action from another logic, method, and/or system to be performed as disclosed herein. Equivalent logic may include firmware, a microprocessor programmed with an algorithm, a discrete logic (e.g., ASIC), at least one circuit, an analog circuit, a digital circuit, a programmed logic device, a memory device containing instructions of an algorithm, and so on, any of which may be configured to perform one or more of the disclosed functions. In one embodiment, logic may include one or more gates, combinations of gates, or other circuit components configured to perform one or more of the disclosed functions. Where multiple logics are described, it may be possible to incorporate the multiple logics into one logic. Similarly, where a single logic is described, it may be possible to distribute that single logic between multiple logics. In one embodiment, one or more of these logics are corresponding structure associated with performing the disclosed and/or claimed functions. Choice of which type of logic to implement may be based on desired system conditions or specifications. For example, if greater speed is a consideration, then hardware would be selected to implement functions. If a lower cost is a consideration, then stored instructions/executable application would be selected to implement the functions. Logic is limited to statutory subject matter under 35 U.S.C. § 101.

An “operable connection”, or a connection by which entities are “operably connected”, is one in which signals, physical communications, and/or logical communications may be sent and/or received. An operable connection may include a physical interface, an electrical interface, and/or a data interface. An operable connection may include differing combinations of interfaces and/or connections sufficient to allow operable control. For example, two entities can be operably connected to communicate signals to each other directly or through one or more intermediate entities (e.g., processor, operating system, logic, non-transitory computer-readable medium). Logical and/or physical communication channels can be used to create an operable connection.

“User”, as used herein, includes but is not limited to one or more persons, computers or other devices, or combinations of these.

While the disclosed embodiments have been illustrated and described in considerable detail, it is not the intention to restrict or in any way limit the scope of the appended claims to such detail. It is, of course, not possible to describe every conceivable combination of components or methodologies for purposes of describing the various aspects of the subject matter. Therefore, the disclosure is not limited to the specific details or the illustrative examples shown and described. Thus, this disclosure is intended to embrace alterations, modifications, and variations that fall within the scope of the appended claims, which satisfy the statutory subject matter requirements of 35 U.S.C. § 101.

To the extent that the term “includes” or “including” is employed in the detailed description or the claims, it is intended to be inclusive in a manner similar to the term “comprising” as that term is interpreted when employed as a transitional word in a claim.

Therefore, provided herein is a new and improved system and method for managing medical devices and medical device consumables, which according to various embodiments of the present invention, offers the following advantages: ease of use; the ability to keep track of medical device installation, usage and maintenance; the ability to keep track of medical device consumable installation, usage and maintenance; the ability to provide billing statements for medical device and medical device consumable usage; the ability to provide feedback regarding the medical device usage and the medical device consumable usage for preventative maintenance; the ability to collect information regarding the medical device usage and the medical device consumable usage for product improvement; the ability to provide audible and haptic (touch) alerts; and the ability to be able to ship, in a timely manner, the needed medical device and/or medical device consumable to the user.

In fact, in many of the preferred embodiments, these advantages of ease of use, the ability to keep track of medical device installation, usage and maintenance, the ability to keep track of medical device consumable installation, usage and maintenance, the ability to provide billing statements for medical device and medical device consumable usage, the ability to provide feedback regarding the medical device usage and the medical device consumable usage for preventative maintenance, the ability to collect information regarding the medical device usage and the medical device consumable usage for product improvement, the ability to provide audible and haptic (touch) alerts, and the ability to be able to ship, in a timely manner, the needed medical device and/or medical device consumable to the user are optimized to an extent that is considerably higher than heretofore achieved in prior, known systems and methods for managing medical devices and medical device consumables.

What is claimed is:

1. A non-transitory computer-readable medium storing computer-executable instructions that, when executed by at least a processor of a computer, cause the computer to:
determine, by at least the processor, if a consumable has been properly installed;
determine, by at least the processor, if the consumable is authentic;
determine, by at least the processor, if the consumable is operating correctly;
determine, by at least the processor, if the consumable needs to be replaced due to a time of usage;
determine, by at least the processor, if the consumable needs to be re-ordered; and
determine, by at least the processor, if a user of the consumable needs to be billed for the use of the consumable.

2. The non-transitory computer-readable medium of claim 1, further comprising instructions that, when executed by at least the processor, cause the computer to:
obtain information from the consumable regarding an operating characteristic of the consumable; and
forward the information to a remote computing device associated with the user.

3. The non-transitory computer-readable medium of claim 1, wherein the determine if a consumable has been properly installed further comprises instructions that, when executed by at least the processor, cause the computer to:
determine, by the at least one processor, if the consumable is installed;
determine, by the at least one processor, if the consumable has been properly installed;
transmit, by the at least one processor, a first notification to the user to inform the user that the consumable has not been properly installed;
display, by the at least one processor, the first notification on a display associated with a remote computing device associated with the user;
upon a maintenance being performed on the consumable, determine, by the at least one processor, if the consumable has been properly installed;
transmit, by the at least one processor, a second notification to the user to inform the user that the consumable has been properly installed; and
display, by the at least one processor, the second notification on the display associated with a remote computing device associated with the user.

4. The non-transitory computer-readable medium of claim 1, wherein the determine if the consumable is authentic further comprises instructions that, when executed by at least the processor, cause the computer to:
determine, by the at least one processor, if the consumable is installed;
determine, by the at least one processor, if the consumable is authentic;
transmit, by the at least one processor, a first notification to the user to inform the user that the consumable is not authentic;
display, by the at least one processor, the first notification on a display associated with a remote computing device associated with the user;
upon an installation of another consumable, determine, by the at least one processor, if the another consumable is authentic;
transmit, by the at least one processor, a second notification to the user to inform the user that the another consumable is authentic; and
display, by the at least one processor, the second notification on the display associated with a remote computing device associated with the user.

5. The non-transitory computer-readable medium of claim 1, wherein the determine if the consumable is operating correctly further comprises instructions that, when executed by at least the processor, cause the computer to:
determine, by the at least one processor, if the consumable is operating;
determine, by the at least one processor, if the consumable is operating correctly;
transmit, by the at least one processor, a first notification to the user to inform the user that the consumable is not operating correctly;
display, by the at least one processor, the first notification on a display associated with a remote computing device associated with the user;
upon completing a maintenance on the consumable, determine, by the at least one processor, if the consumable is operating correctly;
transmit, by the at least one processor, a second notification to the user to inform the user that the consumable is operating correctly; and
display, by the at least one processor, the second notification on the display associated with a remote computing device associated with the user.

6. The non-transitory computer-readable medium of claim 1, wherein the determine if the consumable needs to be replace due to a time of usage further comprises instructions that, when executed by at least the processor, cause the computer to:
determine, by the at least one processor, if the consumable is operating;
determine, by the at least one processor, how long the consumable has been operating;
transmit, by the at least one processor, a first notification to the user to inform the user that the consumable has been operating past a pre-determined period of time;
display, by the at least one processor, the first notification on a display associated with a remote computing device associated with the user;
upon a maintenance being performed on the consumable, determine, by the at least one processor, how long the maintained consumable has been operating;
transmit, by the at least one processor, a second notification to the user to inform the user that the consumable maintenance has been completed; and
display, by the at least one processor, the second notification on the display associated with a remote computing device associated with the user.

7. The non-transitory computer-readable medium of claim 1, wherein the determine if the consumable needs to be re-ordered further comprises instructions that, when executed by at least the processor, cause the computer to:
determine, by the at least one processor, if the consumable has been replaced;
determine, by the at least one processor, an amount of the consumable that is remaining in a consumable inventory;
reduce, by the at least one processor, the amount of consumable that is remaining in the consumable inventory by an amount of the consumable that has been replaced;
upon reducing the amount of consumable that is remaining in the consumable inventory, determine, by the at least one processor, the difference between the amount of consumable that is remaining in the reduced consumable inventory and a threshold amount of consumable that is desired to be stored in the consumable inventory;
place, by the at least one processor, an order for an additional amount of the consumable,
wherein the additional amount of the consumable is equal to the difference between the amount of consumable that is remaining in the reduced consumable inventory and a threshold amount of consumable that is desired to be stored in the consumable inventory;
generate, by the at least one processor, an electronic message that includes an order for the additional amount of the consumable;
transmit, by the at least one processor, via network communications, the electronic message and order to a remote computing system;
receive the electronic message and order and cause a machine to retrieve the additional amount of the consumable from a desired consumable storage location based upon the order;
transport the retrieved additional amount of the consumable; and
place the retrieved additional amount of the consumable in a consumable storage area.

8. A computing system, comprising:
at least one processor connected to at least one memory comprising a non-transitory computer readable medium;
a logic module stored on the memory and including instructions that, when executed by the at least one processor, cause the computing system to:
determine, by at least the processor, if a consumable has been properly installed;
determine, by at least the processor, if the consumable is authentic;
determine, by at least the processor, if the consumable is operating correctly;
determine, by at least the processor, if the consumable needs to be replaced due to a time of usage;
determine, by at least the processor, if the consumable needs to be re-ordered; and
determine, by at least the processor, if a user of the consumable needs to be billed for the use of the consumable.

9. The computing system of claim 8, wherein the logic module further includes instructions that, when executed by the at least one processor, cause the computing system to:
obtain information from the consumable regarding an operating characteristic of the consumable; and
forward the information to a remote computing device associated with the user.

10. The computing system of claim 8, wherein the logic module further includes instructions related to determine if a consumable has been properly installed that, when executed by the at least one processor, cause the computing system to:
determine, by the at least one processor, if the consumable is installed;
determine, by the at least one processor, if the consumable has been properly installed;
transmit, by the at least one processor, a first notification to the user to inform the user that the consumable has not been properly installed;
display, by the at least one processor, the first notification on a display associated with a remote computing device associated with the user;
upon a maintenance being performed on the consumable, determine, by the at least one processor, if the consumable has been properly installed;
transmit, by the at least one processor, a second notification to the user to inform the user that the consumable has been properly installed; and
display, by the at least one processor, the second notification on the display associated with a remote computing device associated with the user.

11. The computing system of claim 8, wherein the logic module further includes instructions related to determine if the consumable is authentic that, when executed by the at least one processor, cause the computing system to:
determine, by the at least one processor, if the consumable is installed;
determine, by the at least one processor, if the consumable is authentic;
transmit, by the at least one processor, a first notification to the user to inform the user that the consumable is not authentic;
display, by the at least one processor, the first notification on a display associated with a remote computing device associated with the user;
upon an installation of an another consumable, determine, by the at least one processor, if the another consumable has been properly installed;
transmit, by the at least one processor, a second notification to the user to inform the user that the another consumable is authentic; and
display, by the at least one processor, the second notification on the display associated with a remote computing device associated with the user.

12. The computing system of claim 8, wherein the logic module further includes instructions related to determine if the consumable is operating that, when executed by the at least one processor, cause the computing system to:
determine, by the at least one processor, if the consumable is operating;
determine, by the at least one processor, if the consumable is operating correctly;
transmit, by the at least one processor, a first notification to the user to inform the user that the consumable is not operating correctly;
display, by the at least one processor, the first notification on a display associated with a remote computing device associated with the user;
upon completing a maintenance on the consumable, determine, by the at least one processor, if the consumable is operating correctly;
transmit, by the at least one processor, a second notification to the user to inform the user that the consumable is operating correctly; and
display, by the at least one processor, the second notification on the display associated with a remote computing device associated with the user.

13. The computing system of claim 8, wherein the logic module further includes instructions related to determine if the consumable needs to be replace due to a time of usage that, when executed by the at least one processor, cause the computing system to:
determine, by the at least one processor, if the consumable is operating;
determine, by the at least one processor, how long the consumable has been operating;
transmit, by the at least one processor, a first notification to the user to inform the user that the consumable has been operating past a pre-determined period of time;
display, by the at least one processor, the first notification on a display associated with a remote computing device associated with the user;
upon a maintenance being performed on the consumable, determine, by the at least one processor, how long the maintained consumable has been operating;
transmit, by the at least one processor, a second notification to the user to inform the user that the consumable maintenance has been completed; and
display, by the at least one processor, the second notification on the display associated with a remote computing device associated with the user.

14. The computing system of claim 8, wherein the logic module further includes instructions relate to determine if the consumable needs to be re-ordered that, when executed by the at least one processor, cause the computing system to:
determine, by the at least one processor, if the consumable has been replaced;
determine, by the at least one processor, an amount of the consumable that is remaining in a consumable inventory;
reduce, by the at least one processor, the amount of consumable that is remaining in the consumable inventory by an amount of the consumable that has been replaced;
upon reducing the amount of consumable that is remaining in the consumable inventory, determine, by the at least one processor, the difference between the amount of consumable that is remaining in the reduced consumable inventory and a threshold amount of consumable that is desired to be stored in the consumable inventory;
place, by the at least one processor, an order for an additional amount of the consumable,
wherein the additional amount of the consumable is equal to the difference between the amount of consumable that is remaining in the reduced consumable inventory and a threshold amount of consumable that is desired to be stored in the consumable inventory;
generate, by the at least one processor, an electronic message that includes an order for the additional amount of the consumable;
transmit, by the at least one processor, via network communications, the electronic message and order to a remote computing system;
receive the electronic message and order and cause a machine to retrieve the additional amount of the consumable from a desired consumable storage location based upon the order;
transport the retrieved additional amount of the consumable; and
place the retrieved additional amount of the consumable in a consumable storage area.

15. A computer-implemented method, the method comprising:
determining if a consumable has been properly installed;
determining if the consumable is authentic;
determining if the consumable is operating correctly;
determining if the consumable needs to be replaced due to a time of usage;
determining if the consumable needs to be re-ordered; and
determining, by at least the processor, if a user of the consumable needs to be billed for the use of the consumable.

16. The method of claim 15, wherein the method further comprises:
obtaining information from the consumable regarding an operating characteristic of the consumable; and
forwarding the information to a remote computing device associated with the user.

17. The method of claim 15, wherein the determining if a consumable has been properly installed method further comprises:
determining if the consumable is installed;
determining if the consumable has been properly installed;
transmitting a first notification to the user to inform the user that the consumable has not been properly installed;
displaying the first notification on a display associated with a remote computing device associated with the user;
upon a maintenance being performed on the consumable, determining if the consumable has been properly installed;
transmitting a second notification to the user to inform the user that the consumable has been properly installed; and
displaying the second notification on the display associated with a remote computing device associated with the user.

18. The method of claim 15, wherein the determining if the consumable is authentic further comprises:
determining if the consumable is installed;
determining if the consumable is authentic;
transmitting a first notification to the user to inform the user that the consumable is not authentic;
displaying the first notification on a display associated with a remote computing device associated with the user;
upon an installation of another consumable, determining if the another consumable has been properly installed;
transmitting a second notification to the user to inform the user that the another consumable is authentic; and
displaying the second notification on the display associated with a remote computing device associated with the user.

19. The method of claim 15, wherein the determining if the consumable is operating correctly further comprises:
determining if the consumable is operating;
determining if the consumable is operating correctly;
transmitting a first notification to the user to inform the user that the consumable is not operating correctly;
displaying the first notification on a display associated with a remote computing device associated with the user;
upon completing a maintenance on the consumable, determining if the consumable is operating correctly;
transmitting a second notification to the user to inform the user that the consumable is operating correctly; and
displaying the second notification on the display associated with a remote computing device associated with the user.

20. The method of claim 15, wherein the determining if the consumable needs to be replaced due to a time of usage further comprises:
determining if the consumable is operating;
determining how long the consumable has been operating;
transmitting a first notification to the user to inform the user that the consumable has been operating past a pre-determined period of time;
displaying the first notification on a display associated with a remote computing device associated with the user;
upon a maintenance being performed on the consumable, determining how long the maintained consumable has been operating;
transmitting a second notification to the user to inform the user that the consumable maintenance has been completed; and
displaying the second notification on the display associated with a remote computing device associated with the user.
